(19) United States
(12) Patent Application Publication
(10) Pub. No.: US 2009/0264716 A1
(43) Pub. Date: Oct. 22, 2009

(54) HEMODYNAMIC MORPHOLOGICAL ANALYSIS FOR RHYTHM IDENTIFICATION

(76) Inventors: Allan C. Shuros, St. Paul, MN (US); Dan Li, Shoreview, MN (US)

Correspondence Address: SCHWEGMAN, LUNDBERG & WOESSNER/BSC-CRM, PO BOX 2938, MINNEAPOLIS, MN, 55402, US

(21) Appl. No.: 12/425,193

(22) Filed: Apr. 16, 2009

Related U.S. Application Data
(60) Provisional application No. 61/045,922, filed on Apr. 17, 2008.

(51) Int. Cl. A61N 1/365; A61B 5/0205; A61B 5/00; A61N 1/39

(52) U.S. Cl. 600/301; 600/483; 607/6; 607/14

(57) ABSTRACT

This document discusses, among other things, an apparatus comprising an implantable hemodynamic sensor circuit that provides a hemodynamic signal representative of mechanical function of a cardiovascular system of a subject and a controller circuit communicatively coupled to the hemodynamic sensor circuit. The controller circuit includes a detection module configured to detect an onset of tachyarrhythmia, a signal analyzer module configured to determine a measure of morphological variability of the hemodynamic signal during the episode of tachyarrhythmia, and a rhythm discrimination module configured to deem whether the tachyarrhythmia episode is indicative of ventricular tachycardia (VT) according to the measure of morphological variability.

### RELATED APPLICATIONS

This application claims the benefit under 35 U.S.C. 119(e) of U.S. Provisional Patent Application Ser. No. 61/045,922, filed on Apr. 17, 2008, which is incorporated herein by reference in it entirety.

### BACKGROUND

Implantable medical devices (IMDs) include devices designed to be implanted into a patient. Some examples of these devices include cardiac function management (CFM) devices such as implantable pacemakers, implantable cardioverter defibrillators (ICDs), cardiac resynchronization therapy devices (CRTs), and devices that include a combination of such capabilities. The devices can be used to treat patients using electrical or other therapy or to aid a physician or caregiver in patient diagnosis through internal monitoring of a patient's condition. The devices may include one or more electrodes in communication with one or more sense amplifiers to monitor electrical heart activity within a patient, and often include one or more sensors to monitor one or more other internal patient parameters. Other examples of implantable medical devices include implantable diagnostic devices, implantable drug delivery systems, or implantable devices with neural stimulation capability.

Additionally, some IMDs detect events by monitoring electrical heart activity signals. In CFM devices, these events can include heart chamber expansions or contractions. By monitoring cardiac signals indicative of expansions or contractions, IMDs can detect abnormally slow heart rate, or bradycardia. Some IMDs detect abnormally rapid heart rate, or tachyarrhythmia. Tachyarrhythmia includes ventricular tachycardia (VT) and supraventricular tachycardia (SVT). Tachyarrhythmia also includes rapid and irregular heart rate, or fibrillation, including ventricular fibrillation (VF).

When detected, tachyarrhythmia can be terminated with high energy shock therapy using an ICD. Under-detection of tachyarrhythmia (i.e., the IMD does not recognize an episode of tachyarrhythmia) may leave tachyarrhythmia untreated. Additionally, over-detection of tachyarrhythmia by the IMD (i.e., the IMD categorizes too many false-positives as tachyarrhythmia) is undesirable for the patient and the device. Cardioversion/defibrillation therapy can cause patient discomfort and consumes a relatively large amount of battery power which may lead to a shortened useful device lifetime. Therefore, it is important to accurately detect tachyarrhythmia.

### Overview

This document relates generally to systems, devices, and methods for monitoring hemodynamic parameters of a patient or subject. In example 1, an apparatus includes an implantable hemodynamic sensor circuit that provides a hemodynamic signal representative of mechanical function of a cardiovascular system of a subject and a controller circuit communicatively coupled to the hemodynamic sensor circuit. The controller circuit includes a detection module configured to detect an onset of tachyarrhythmia, a signal analyzer module configured to determine a measure of morphological variability of the hemodynamic signal during the episode of tachyarrhythmia, and a rhythm discrimination module configured to deem whether the tachyarrhythmia episode is indicative of ventricular tachycardia VT according to the measure of morphological variability.

In example 2, the signal analyzer module of example 1 is optionally configured to compare a morphological feature of a sensed hemodynamic signal to a hemodynamic signal template, and determine a measure of morphological variability of the hemodynamic signal using the comparison.

In example 3, the signal analyzer module of examples 1-2 is optionally configured to determine a correlation value that indicates whether the hemodynamic signal correlates to the hemodynamic template signal, determine a measure of variability of the correlation value, and the rhythm discrimination module of the examples is optionally configured to deem whether the onset of tachyarrhythmia is indicative of ventricular tachycardia according to the measure of variability of the correlation value.

In example 4, the apparatus of examples 1-3 optionally includes an implantable cardiac signal sensing circuit, communicatively coupled to the controller circuit, configured to obtain a sensed electrical cardiac signal associated with an action potential of the patient's heart. The controller circuit of the examples optionally includes a template module configured to determine a fiducial identifier for alignment of a plurality of sensed hemodynamic signals using the sensed electrical cardiac signal, and generate the hemodynamic signal template using a central tendency of aligned sensed hemodynamic signals.

In example 5, the template module of example 4 is optionally configured to identify a candidate hemodynamic signal for inclusion in the template generating using at least one rule, and discriminate, according to the rule, whether the candidate hemodynamic signal is representative of cardiac mechanical function during an intrinsic depolarization.

In example 6, the apparatus of examples 1-5 optionally includes an implantable cardiac signal sensing circuit (communicatively coupled to the controller circuit and configured to obtain a sensed electrical cardiac signal associated with an action potential of the patient's heart), a sampling circuit configured to obtain sampled data from the sensed hemodynamic signal and the sensed electrical cardiac signal, and a communication circuit for communicating information with an external device. The controller circuit of the examples is communicatively coupled to the sampling circuit and the communication circuit and is configured to communicate the sampled data to an external device, and receive the hemodynamic signal template from the external device via the communication circuit.

In example 7, the signal analyzer module of examples 1-6 is optionally configured to determine a measure of variability of a time duration between two different fiducial identifiers in the hemodynamic signal, determine a measure of morphological complexity of the hemodynamic signal during the episode of tachyarrhythmia. The rhythm discrimination module of the examples is optionally configured to deem whether the onset of tachyarrhythmia is indicative of ventricular tachycardia according to both the measure of variability of the time duration and the measure of morphological complexity.

In example 8, the signal analyzer module of examples 1-7 is optionally configured to measure variability of at least one of a maximum of the hemodynamic signal, a minimum of the hemodynamic signal, a maximum rate of change of the hemodynamic signal, a minimum rate of change of the hemodynamic signal, a time where the hemodynamic signal reaches a determined maximum, a time where the hemodynamic signal reaches a determined minimum, a time of occurrence of an inflection point in the hemodynamic signal, an area under a curve of a segment of the hemodynamic signal, or an Nth moment of the hemodynamic signal.

In example 9, the signal analyzer module of examples 1-8 is configured to determine a first fiducial identifier and a second fiducial identifier in the hemodynamic signal, and obtain a measure of variability of a time duration between the first and second fiducial identifiers.

In example 10, the signal analyzer module of examples 1-9 is optionally configured to obtain a transform of the hemodynamic signal from a time domain to a frequency domain, and to measure a variability of at least one of a dominant frequency of the transformed hemodynamic signal, or a magnitude of a power spectral density of the transformed hemodynamic signal.

In example 11, the signal analyzer module of examples 1-10 optionally includes an implantable cardiac depolarization sensing circuit communicatively coupled to the controller circuit and configured to obtain a sensed depolarization signal from a ventricle. The detection module of the examples is optionally configured to monitor a rate of ventricular depolarizations of the subject using the sensed depolarization signal, and detect the tachyarrhythmia episode when detecting a rate of depolarizations that exceeds a ventricular tachycardia detection rate threshold for a specified time duration.

In example 12, the hemodynamic sensor circuit of examples 1-11 optionally includes an intracardiac impedance sensing circuit, and the hemodynamic signal is representative of intracardiac impedance of the subject.

In example 13, the hemodynamic sensor circuit of examples 1-12 optionally includes a transthoracic impedance sensing circuit, and the hemodynamic signal is representative of a cardiac component of transthoracic impedance of the subject.

In example 14, the hemodynamic sensor circuit of examples 1-13 optionally includes a cardiac pressure sensing circuit, and the hemodynamic signal is representative of cardiac pressure.

In example 15, the cardiac pressure sensing circuit of example 14 optionally includes at least one of a pressure sensor to be implanted in a coronary vessel to determine left ventricle pressure by direct measurement of coronary vessel pressure, a right ventricle (RV) chamber pressure sensor, a pulmonary artery pressure sensor, a left atrial chamber pressure sensor, or a pressure sensor to be implanted in a vein near an artery to determine arterial pressure from contact with the artery.

In example 16, the hemodynamic sensor circuit of examples 1-14 optionally includes at least one of a heart sound sensor, a cardiac wall motion sensor, or a supracardiac impedance sensing circuit.

In example 17, the apparatus of examples 1-16 optionally includes a therapy circuit communicatively coupled to the controller circuit and configured to provide at least one of pacing therapy, cardioversion therapy, or defibrillation therapy. The rhythm discrimination module of the examples is optionally configured to deem whether the onset of tachyarrhythmia is indicative of VT or supraventricular tachycardia (SVT) according to the measure of variability. The controller circuit of the examples is configured to initiate at least one of anti-tachycardia pacing (ATP) therapy, cardioversion therapy, or defibrillation therapy, according to the measure of morphological variability when the onset is deemed to VT.

In example 18, a method includes using an implantable sensor to sense a hemodynamic signal that is an electrical signal representative of mechanical function of a cardiovascular system of a subject, detecting an onset of tachyarrhythmia, determining a measure of morphological variability of the hemodynamic signal during the episode of tachyarrhythmia, and deeming whether the onset of tachyarrhythmia is indicative of VT according to the measure of morphological variability.

In example 19, the determining a measure of morphological variability of example 18 optionally includes comparing morphological features of a sensed hemodynamic signal to a hemodynamic signal template, and determining a measure of morphological variability of the hemodynamic signal during the onset of tachyarrhythmia using the comparison.

In example 20, the determining a measure of morphological variability of examples 18-19 optionally includes determining a correlation value to indicate whether the hemodynamic signal correlates to the hemodynamic signal template, determining a measure of variability of the correlation value, and deeming whether the onset of tachyarrhythmia is indicative of ventricular tachycardia according to the measure of variability of the correlation value.

In example 21, the method of examples 18-20 optionally includes sensing an electrical cardiac signal associated with an action potential of the subject's heart, determining a fiducial identifier for alignment of a plurality of sensed hemodynamic signals using the sensed electrical cardiac signal, and generating the hemodynamic signal template using a central tendency of aligned sensed hemodynamic signals.

In example 22, the method of examples 18-21 optionally includes identifying a candidate hemodynamic signal, using at least one rule, for inclusion in generating the hemodynamic signal template, and the rule discriminates whether the candidate hemodynamic signal is representative of cardiac mechanical function during an intrinsic depolarization.

In example 23, the determining a measure of morphological variability of examples 18-22 optionally includes determining a measure of variability of a cycle length of the hemodynamic signal, determining a measure of morphological complexity of the hemodynamic signal during the episode of tachyarrhythmia, and deeming whether the onset of tachyarrhythmia is indicative of ventricular tachycardia according to both the measure of variability of the cycle length and the measure of morphological complexity.

In example 24, the detecting a tachyarrhythmia episode of examples 18-23 optionally includes monitoring a rate of ventricular depolarizations of the subject, and detecting that an increase in the rate of ventricular depolarizations exceeds a ventricular tachycardia detection rate threshold for a specified time duration.

In example 25, the sensing a hemodynamic signal of examples 18-24 optionally includes sensing a signal representative of at least one of intracardiac impedance, or a cardiac component of transthoracic impedance.

In example 26, the sensing a hemodynamic signal of examples 18-25 optionally includes sensing a signal representative of cardiac pressure.

In example 27, the sensing a signal representative of cardiac pressure of example 26 optionally includes sensing a signal representative of at least one of, right ventricular pressure, pulmonary arterial pressure, right atrial pressure, left atrial pressure, left ventricular pressure, left ventricular pressure as sensed in a coronary vessel, or an arterial pressure.

In example 28, the sensing a hemodynamic signal of examples 18-27 optionally includes sensing at least one of a heart sound associated with activity of the subject's heart, an electrical signal representative of ventricular wall motion, or a signal representative of supracardiac impedance.

In example 29, the determining a measure of morphological variability of examples 18-28 optionally includes measuring variability of a hemodynamic signal feature that includes at least one of a maximum of the hemodynamic signal, a minimum of the hemodynamic signal, a maximum rate of change of the hemodynamic signal, a minimum rate of change of the hemodynamic signal, a time where the hemodynamic signal reaches a determined maximum, a time where the hemodynamic signal reaches a determined minimum, a time of occurrence of an inflection point in the hemodynamic signal, an area under a curve identified in a segment of the hemodynamic signal, or an Nth moment of the hemodynamic signal.

In example 30, the determining a measure of morphological variability of examples 18-29 optionally includes determining a first fiducial identifier and a second fiducial identifier in the sensed hemodynamic signals, and measuring the variability of a time duration between the first and second fiducial identifiers.

In example 31, the determining a measure of morphological variability of examples 18-30 optionally includes obtaining a transform of the hemodynamic signal from a time domain to a frequency domain, and determining a measure of variability of at least one of a dominant frequency of the transformed hemodynamic signal, or a magnitude of a power spectral density of the transformed hemodynamic signal.

In example 32, the deeming whether the onset of tachyarrhythmia is indicative of VT according to the measure of variability of examples 18-31 optionally includes discriminating whether the onset of tachyarrhythmia is indicative of VT or SVT, and the method optionally includes initiating ATP therapy when the tachyarrhythmia episode is deemed to be SVT and initiating at least one of cardioversion therapy or defibrillation therapy when the tachyarrhythmia episode is deemed to be VT.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of a system that uses an IMD.

FIG. 2 illustrates an example of morphology of a hemodynamic signal.

FIG. 3 is an illustration of the signals of FIG. 2 during induced ventricular tachycardia.

FIG. 4 is a flow diagram of a method of identifying tachyarrhythmia using an IMD.

FIG. 5 shows a block diagram of an example of an IMD that identifies tachyarrhythmia.

FIG. 6 shows a block diagram of another example of an IMD that identifies tachyarrhythmia.

FIG. 7 shows a block diagram of still another example of an IMD that identifies tachyarrhythmia.

### DETAILED DESCRIPTION

An IMD may include one or more of the features, structures, methods, or combinations thereof described herein. For example, a cardiac monitor or a cardiac stimulator may be implemented to include one or more of the advantageous features and/or processes described below. It is intended that such a monitor, stimulator, or other implantable or partially implantable device need not include all of the features described herein, but may be implemented to include selected features that provide for unique structures and/or functionality. Such a device may be implemented to provide a variety of therapeutic or diagnostic functions.

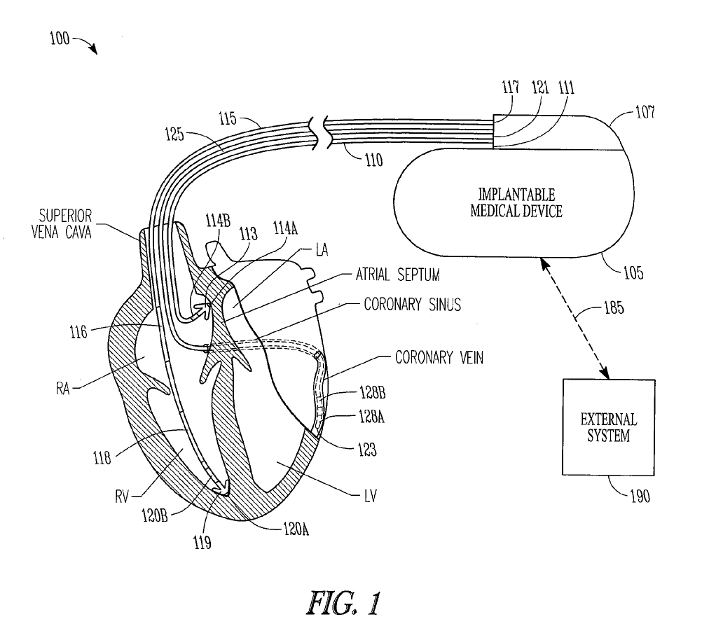

FIG. 1 is an illustration of portions of a system 100 that uses an IMD 105. Examples of IMD 105 include, without limitation, a pacemaker, a cardioverter, a defibrillator, a cardiac resynchronization therapy (CRT) device, and other cardiac monitoring and therapy delivery devices, including cardiac devices that include or work in coordination with one or more neuro-stimulating devices, drugs, drug delivery systems, or other therapies. As one example, the system 100 shown is used to treat a cardiac arrhythmia. The IMD 105 typically includes an electronics unit coupled by one or more cardiac leads 110, 115, 125, to a heart of a patient or subject. The electronics unit of the IMD 105 typically includes components that are enclosed in a hermetically-sealed canister or “can.” The system 100 also typically includes an IMD programmer or other external system 190 that communicates one or more wireless signals 185 with the IMD 105, such as by using radio frequency (RF) or by one or more other telemetry methods.

The example shown includes right atrial (RA) lead 110 having a proximal end 111 and a distal end 113. The proximal end 111 is coupled to a header connector 107 of the IMD 105. The distal end 113 is configured for placement in the RA in or near the atrial septum. The RA lead 110 may include a pair of bipolar electrodes, such as an RA tip electrode 114A and an RA ring electrode 114B. The RA electrodes 114A and 114B are incorporated into the lead body at distal end 113 for placement in or near the RA, and are each electrically coupled to IMD 105 through a conductor extending within the lead body. The RA lead is shown placed in the atrial septum, but the RA lead may be placed in or near the atrial appendage, the atrial free wall, or elsewhere.

The example shown also includes a right ventricular (RV) lead 115 having a proximal end 117 and a distal end 119. The proximal end 117 is coupled to a header connector 107. The distal end 119 is configured for placement in the RV. The RV lead 115 may include one or more of a proximal defibrillation electrode 116, a distal defibrillation electrode 118, an RV tip electrode 120A, and an RV ring electrode 120B. The defibrillation electrode 116 is generally incorporated into the lead body such as in a location suitable for supraventricular placement in the RA and/or the superior vena cava. The defibrillation electrode 118 is incorporated into the lead body near the distal end 119 such as for placement in the RV. The RV electrodes 120A and 120B may form a bipolar electrode pair and are generally incorporated into the lead body at distal end 119. The electrodes 116, 118, 120A, and 120B are each electrically coupled to IMD 105, such as through one or more conductors extending within the lead body. The proximal defibrillation electrode 116, distal defibrillation electrode 118, or an electrode formed on the can of IMD 105 allow for delivery of cardioversion or defibrillation pulses to the heart.

The RV tip electrode 120A, RV ring electrode 120B, or an electrode formed on the can of IMD 105 allow for sensing an RV electrogram signal representative of RV depolarizations and delivering RV pacing pulses. In some examples, the IMD includes a sense amplifier circuit to provide amplification and/or filtering of the sensed signal. RA tip electrode 114A, RA ring electrode 114B, or an electrode formed on the can of IMD 105 allow for sensing an RA electrogram signal representative of RA depolarizations and allow for delivering RA pacing pulses. Sensing and pacing allows the IMD 105 to adjust timing of the heart chamber contractions. In some examples, the IMD 105 can adjust the timing of ventricular depolarizations with respect to the timing of atrial depolarizations by sensing electrical signals in the RA and pacing the RV at the desired atrial-ventricular (AV) delay time.

A left ventricular (LV) lead 125 can include a coronary pacing or sensing lead that includes an elongate lead body having a proximal end 121 and a distal end 123. The proximal end 121 is coupled to a header connector 107. A distal end 123 is configured for placement or insertion in the coronary vein. The LV lead 125 may include an LV ring or tip electrode 128A and an LV ring electrode 128B. The distal portion of the LV lead 125 is configured for placement in the coronary sinus and coronary vein such that the LV electrodes 128A and 128B are placed in the coronary vein. The LV electrodes 128A and 128B may form a bipolar electrode pair and are typically incorporated into the lead body at distal end 123. Each can be electrically coupled to IMD 105 such as through one or more conductors extending within the lead body. LV tip electrode 128A, LV ring electrode 128B, or an electrode formed on the can of the IMD 105 allow for sensing an LV electrogram signal representative of LV depolarizations and delivering LV pacing pulses.

The IMDs may be configured with a variety of electrode arrangements, including transvenous, epicardial electrodes (i.e., intrathoracic electrodes), and/or subcutaneous, non-intrathoracic electrodes, including can, header, and indifferent electrodes, and subcutaneous array or lead electrodes (i.e., non-intrathoracic electrodes).

Episodes of tachyarrhythmia may be detected using a tiered approach. In a first tier of detection, a possible episode is first identified when a sensed heart depolarization rate exceeds a specified tachyarrhythmia heart rate threshold. In a second tier of detection, techniques are applied to sensed intrinsic cardiac signals to determine if the detected rhythm is abnormal. These intrinsic cardiac signals are electrical signals associated with action potentials of the heart. The action potentials propagate through the heart's electrical conduction system to excite various regions of myocardial tissue. In an example, an abnormal cardiac rhythm can be detected using an assessment of heart rhythm stability when a subject experiences a sudden increase in heart rate. Examples of methods and systems to detect abnormal heart rhythms and assess the stability of the rhythms are found in Gilkerson et al., U.S. Pat. No. 6,493,579, entitled “System and Method for Detection Enhancement Programming,” filed Aug. 20, 1999, which is incorporated herein by reference in its entirety.

Hemodynamic information can also be used to identify tachyarrhythmia. More specifically, a hemodynamic morphological analysis of the patient or subject can provide rhythm discrimination for detecting tachyarrhythmia. Such an analysis can also be used during a first tier or a second tier of rhythm discrimination.

The hemodynamic information is obtained from sensors that monitor mechanical function of the heart. Examples include, among other things, sensors to monitor pressure, intracardiac impedance, heart sounds, heart valve motion, and ventricular wall motion. The sensors provide hemodynamic signals that represent the mechanical functionality of the cardiovascular system. It should be noted this is different from sensing electrical intrinsic cardiac signals which are the action potentials that propagate through the heart's electrical conduction system. Morphological changes to a sensed hemodynamic signal may provide an indication that the subject is experiencing tachyarrhythmia. An analysis of the morphological changes may then be used to determine if the subject is experiencing ventricular tachycardia.

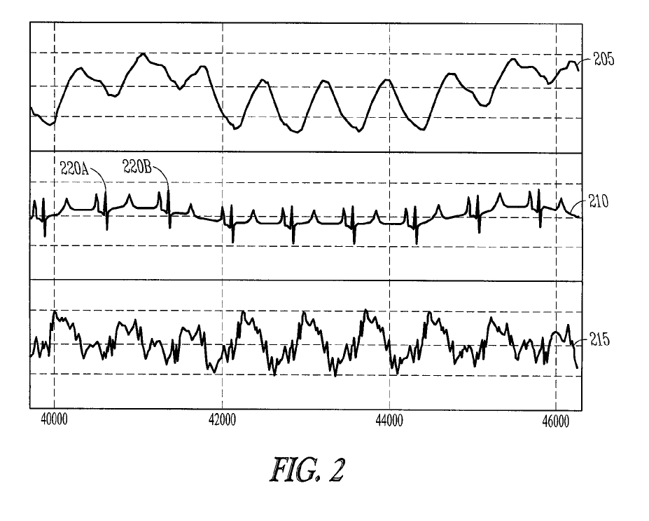

FIG. 2 illustrates an example of morphology of a hemodynamic signal. In this example, the sensed hemodynamic signal represents intracardiac impedance for a normal sinus rhythm (NSR). An intracardiac impedance signal 205 can be obtained using electrodes placed within the heart to provide a signal of impedance versus time. For example, in FIG. 1 a cardiac impedance sensor can sense intracardiac impedance of the right ventricle between an electrode placed at the apex of the right ventricle and an electrode placed in the superior vena cava (SVC). A predetermined excitation current is delivered between the electrodes and the impedance is determined from a voltage sensed between the electrodes. Systems and methods to measure intracardiac impedance are described in Citak et al., U.S. Pat. No. 4,773,401, entitled “Physiologic Control of Pacemaker Rate Using Pre-Ejection Interval as the Controlling Parameter,” filed Aug. 21, 1987, which is incorporated herein by reference.

The example shown also illustrates an electrocardiogram (ECG) signal 210. Also illustrated is a rate of change of intracardiac impedance (dZ/dt) signal 215. During NSR, the intracardiac impedance signal exhibits regularity from cardiac cycle to cardiac cycle. The ECG signal 210 may be useful to assess the regularity of the sensed hemodynamic signal. For example, R-waves 220A, 220B can be used as a fiducial identifier to indicate the cardiac cycles. During tachyarrhythmia, regularity of features in a hemodynamic signal may decrease, or conversely, variability of features in the signal may increase.

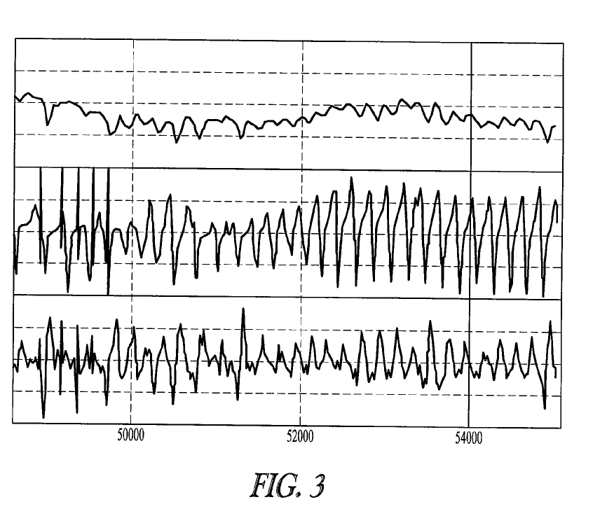

FIG. 3 is an illustration of the signals of FIG. 2 during induced ventricular tachycardia. It can be seen that the signals have become much more irregular. Variability of the intracardiac impedance may be determined using feature points. Examples of feature points include, among other things, the maximum impedance value or the minimum impedance value. In some examples, the variability of the intracardiac impedance is determined by monitoring the variability of the maximum value of dZ/dt or the minimum value of dZ/dt.

Compared to methods that classify tachyarrhythmia using measurements of extracted features of the sensed hemodynamic signal alone, assessing the variability or regularity of the sensed hemodynamic signal during the arrhythmia may disclose subtle changes in hemodynamic system variation that may otherwise be difficult to detect.

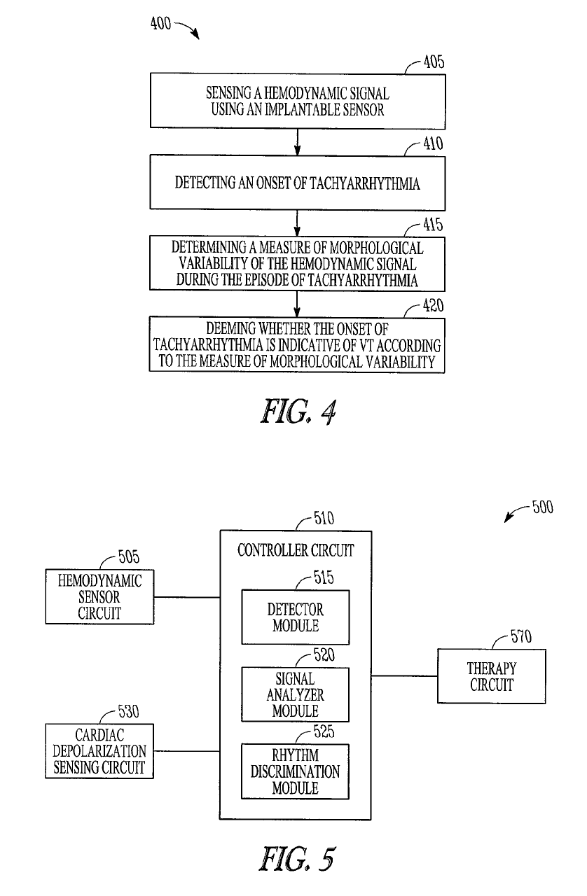

FIG. 4 is a flow diagram of a method 400 of identifying tachyarrhythmia using an IMD. At block 405, a hemodynamic signal is sensed using an implantable sensor. As discussed previously, the implantable sensor provides an electrical signal representative of mechanical function of a cardiovascular system of a subject. The implantable sensor may monitor, among other things, pressure, intracardiac impedance, heart sounds, heart valve motion, and ventricular wall motion.

At block 410, an onset of tachyarrhythmia is detected by the IMD. In some examples, the IMD detects the onset when a monitored rate of ventricular depolarizations exceeds a ventricular tachycardia detection rate threshold for a specified time duration. This rate threshold defines the arrhythmia detection zones. However, the IMD may detect the onset without comparing a monitored depolarization rate to a lowest tachyarrhythmia heart rate value. For example, the IMD may detect the onset of tachyarrhythmia when the ventricular depolarization rate exceeds the atrial depolarization rate. In another example, the IMD may detect the onset from a sudden rate increase. Descriptions of systems, devices, and methods to detect tachyarrhythmia without a comparison to a specified lowest tachyarrhythmia rate are found in Kim et al., U.S. Patent Publication No. 20070135848, “Zoneless Tachyarrhythmia Detection with Real Time Rhythm Monitoring, filed Dec., 13, 2005, which is incorporated herein in its entirety.

At block 415, a measure of morphological variability of the hemodynamic signal is determined during the episode of tachyarrhythmia. In some examples, the measure of morphological variability includes the variability of a feature point or other fiducial of the sensed hemodynamic signal. Examples of a measure of variability include, among other things, a variance of the fiducial or a standard deviation of the fiducial. At block 420, the IMD deems whether the onset of tachyarrhythmia is indicative of VT according to the measure of morphological variability. For example, the IMD may deem that the tachyarrhythmia is VT when the determined measure of variability exceeds a specified threshold value.

In some examples, if the IMD determines that the tachyarrhythmia is VT, the IMD provides therapy to the subject. Some IMDs, such as implantable cardioverter defibrillators (ICDs), treat tachycardia by delivering a high energy electrical shock to the heart. Other IMDs provide anti-tachycardia pacing (ATP). ATP uses lower energy pacing energy to establish a regular rhythm in a heart. This allows the tachycardia to be converted to a normal heart rhythm without exposing the patient to high energy defibrillation therapy that can be painful to the patient. Some IMDs are able to provide both ATP and defibrillation. When tachycardia is detected, the device may try to convert the arrhythmia with ATP before resorting to high energy defibrillation.

If the IMD determines the tachyarrhythmia is not VT (e.g., NSR or SVT), the IMD may not provide therapy to the subject. For example, the IMD may determine a measure of variability for the hemodynamic signal that does not exceed the specified threshold value. This may be because the tachyarrhythmia is SVT and the hemodynamic signal exhibits enough stability and/or regularity so that the variability measure does not exceed the specified threshold. In some examples, the IMD may initiate ATP therapy when the tachyarrhythmia is deemed to be SVT.

FIG. 5 shows a block diagram of an example of an IMD 500 that identifies tachyarrhythmia. The IMD 500 includes an implantable hemodynamic sensor circuit 505. The hemodynamic sensor circuit 505 provides a hemodynamic signal representative of mechanical function of a cardiovascular system of a subject. In some examples, the hemodynamic sensor circuit 505 includes an intracardiac impedance sensing circuit, and the hemodynamic signal provided by the intracardiac impedance sensing circuit is representative of intracardiac impedance of the subject.

In some examples, the hemodynamic sensor circuit 505 includes a supracardiac impedance sensing circuit, such as a transthoracic impedance sensing circuit. In a transthoracic impedance sensing circuit, the hemodynamic signal is representative of a cardiac component of transthoracic impedance of the subject. A predetermined excitation current is delivered between the electrodes and the impedance is determined from a voltage sensed between the electrodes. In FIG. 1, a transthoracic impedance of a subject can be measured between the RV ring electrode 120B and an electrode formed on the can of the IMD 105 or an electrode formed on the header connector 107. An approach to measuring transthoracic impedance is described in Hartley et al., U.S. Pat. No. 6,076,015 “Rate Adaptive Cardiac Rhythm Management Device Using Transthoracic Impedance,” filed Feb. 27, 1998, which is incorporated herein by reference.

According to some examples, the hemodynamic sensor circuit 505 includes a cardiac pressure sensing circuit, and the hemodynamic signal is representative of cardiac pressure. An implantable cardiac pressure sensor measures chamber pressure of the left ventricle. In an example, a pressure sensor is implanted in a coronary vessel to determine left ventricle pressure by direct measurement of coronary vessel pressure. An approach to measuring cardiac pressure using an implantable pressure sensor is found in Salo et al., U.S. Pat. No. 6,666,826, entitled “Method and Apparatus for Measuring Left Ventricular Pressure,” filed Jan. 4, 2002, which is incorporated herein by reference. Other cardiac pressure sensors examples include a right ventricle (RV) chamber pressure sensor, a pulmonary artery pressure sensor, and a left atrial chamber pressure sensor, or a pressure sensor to be implanted in a vein near an artery to determine arterial pressure from contact with the artery.

In some examples, the hemodynamic sensor circuit 505 includes a heart sound sensor circuit. Heart sounds are associated with mechanical vibrations from activity of a patient's heart and the flow of blood through the heart. Heart sounds recur with each cardiac cycle and are separated and classified according to the activity associated with the vibration. The first heart sound (S1) is the vibrational sound made by the heart during tensing of the mitral valve. The second heart sound (S2) marks the beginning of diastole. The third heart sound (S3) and fourth heart sound (S4) are related to filling pressures of the left ventricle during diastole. The hemodynamic signal provided by the heart sound sensor is representative of mechanical activity of a patient's heart.

The heart sound sensor is disposed in a heart, or near the heart in a location where the acoustic energy can be sensed. In some examples, the heart sound sensor includes an accelerometer disposed in or near a heart. In another embodiment, the heart sound sensor includes a microphone disposed in or near a heart. An approach for monitoring heart sounds is found in Siejko et al., U.S. Patent Application Publ. No. 2004/0127792, entitled “Method and Apparatus for Monitoring of Diastolic Hemodynamics,” filed Dec. 30, 2002, which is incorporated herein by reference.

In some examples, the hemodynamic sensor circuit 505 includes a cardiac wall motion sensor. An implantable cardiac wall motion sensor detects a reduction in ventricular contractility. Examples of such sensors measure cardiac wall motion using heart sounds, acceleration signals, and/or cardiac impedance. In an example, an accelerometer can be used to provide acceleration signals each indicative of regional cardiac wall motion. One or more accelerometers can be incorporated into a portion of a lead positioned on or in the heart. The accelerometers detect a wall motion abnormality as an abrupt decrease in the amplitude of local cardiac accelerations. An approach to sensing wall motion is found in the commonly assigned, co-pending U.S. patent application Ser. No. 11/135,985, entitled “Systems and Methods for Multi-Axis Cardiac Vibration Measurements,” filed May 24, 2005, which is incorporated herein by reference.

The IMD 500 also includes a controller circuit 510 communicatively coupled to the hemodynamic sensor circuit 505. The communicative coupling allows electrical signals to be communicated between the controller circuit 510 and the hemodynamic sensor circuit 505 even though there may be intervening circuitry between the controller circuit 510 and the hemodynamic sensor circuit 505.

The controller circuit 510 may include a digital signal processor, application specific integrated circuit (ASIC), microprocessor, or other type of processor, interpreting or executing instructions in software or firmware. In some examples, the controller circuit 510 may include a state machine or sequencer that is implemented in hardware circuits. The controller circuit 510 may include any combination of hardware, firmware, or software, and may include one or more modules to perform the functions described herein.

To provide the functions described herein, the controller circuit 510 includes modules. A module may include software, hardware, firmware or any combination thereof. For example, the module may include instructions in software executing on or interpreted by the controller circuit 510. Multiple functions may be performed by one or more modules.

The controller circuit 510 includes a detection module 515, a signal analyzer module 520, and a rhythm discrimination module 525. The detection module 515 detects an onset of tachyarrhythmia. In some examples, the detection module 515 detects tachyarrhythmia using depolarization rate. In the examples, the detection module 515 includes an implantable cardiac depolarization sensing circuit 530 communicatively coupled to the controller circuit 510. In certain examples, the cardiac depolarization sensing circuit 530 obtains a sensed depolarization signal from a ventricle. The detection module 515 monitors a rate of ventricular depolarizations of the subject using the sensed depolarization signal, and detects the tachyarrhythmia episode when detecting a rate of depolarizations that exceeds a ventricular tachycardia detection rate threshold for a specified time duration. In some examples, the detection module 515 detects tachyarrhythmia without a comparison to depolarization rate, such as when a detected atrial depolarization rate exceeds a detected ventricular depolarization rate.

The signal analyzer module 520 determines a measure of morphological variability of the hemodynamic signal during the episode of tachyarrhythmia, and the rhythm discrimination module 525 deems whether the tachyarrhythmia episode is indicative of VT according to the measure of morphological variability.

According to some examples, the signal analyzer module 520 determines the variability of a feature point or fiducial identifier in the sensed hemodynamic signal. The measure of variability may be, among other things, a calculated standard deviation or a calculated variance of the fiducial identifier. In some examples, the fiducial identifier includes one or more of a maximum value of the hemodynamic signal, a minimum value of the hemodynamic signal, a time where the hemodynamic signal reaches the determined maximum, the time where the hemodynamic signal reaches a determined minimum, and a time of occurrence of an inflection point in the hemodynamic signal. An inflection point refers to a point where a curvature of the sensed hemodynamic signal changes sign.

According to some examples, the signal analyzer module 520 determines a first fiducial identifier and a second fiducial identifier in the sensed hemodynamic signals, and measures the variability of a time duration between the first and second fiducial identifiers. In an illustrative example, the first identifier is a maximum value of the hemodynamic signal and the second identifier is a minimum value of the hemodynamic signal. The signal analyzer module 520 measures the variability of the time duration between the maximum and minimum values.

In some examples, the signal analyzer module 520 determines variability of a measured feature of the hemodynamic signal. In certain examples, the signal analyzer module 520 determines variability of one or more of a maximum rate of change of the hemodynamic signal, a minimum rate of change of the hemodynamic signal, an area under a curve of a segment of the hemodynamic signal, and an Nth moment of the hemodynamic signal.

According to some examples, the signal analyzer module 520 determines variability of a measured feature of the hemodynamic signal in the frequency domain. The signal analyzer module 520 obtains a transform of the hemodynamic signal from a time domain to a frequency domain, such as by calculating a Fourier transform of the hemodynamic signal for example. The signal analyzer module 520 determines variability of a measured feature of the hemodynamic signal transform. In certain examples, the signal analyzer module 520 determines variability of a dominant frequency of the transformed hemodynamic signal. In certain examples, the signal analyzer module 520 determines variability of a magnitude of a power spectral density of the transformed hemodynamic signal.

In some examples, the signal analyzer module 520 determines a measure of variability of a time duration between two different fiducial identifiers in the sensed hemodynamic signal (such as two fiducial identifiers to identify the cycle lengths of the intracardiac impedance signal of FIG. 2 for example). The signal analyzer module 520 determines a measure of morphological complexity of the sensed hemodynamic signal during the episode of tachyarrhythmia. The rhythm discrimination module 525 deems whether the onset of tachyarrhythmia is indicative of ventricular tachycardia according to both the measure of variability of the time duration and the measure of morphological complexity.

An example of a measure of variability of cycle lengths is a measure of irregularity sample entropy calculated to provide an indication of the irregularity the cycle length of the hemodynamic signal. A sensed ECG signal (such as ECG signal 210 of FIG. 2) can be used to provide identifiers to monitor the cycle length. An example of a complexity parameter is a measure of complexity sample entropy calculated to provide an indication of the morphological complexity of the hemodynamic signal. An approach to determining tachyarrhythmia using the variability of cycle lengths and the morphological complexity of a sensed electrical cardiac signal is found in Li, U.S. Patent Application publication No. 20060281999, “Method and Apparatus for Cardiac Arrhythmia Classification Using Sample Entropy,” filed Jun. 13, 2005, which is incorporated herein in its entirety.

In certain examples, variability of the sensed hemodynamic signal may be calculated from one segment of the signal to the next. According to some examples, variability of the signal is determined by comparing a segment of the sensed signal to a template of the sensed signal. The signal analyzer module 520 compares a morphological feature of a sensed hemodynamic signal to a hemodynamic signal template, and determines a measure of morphological variability of the hemodynamic signal using the comparison.

The hemodynamic signal template may be a representative signal output from the hemodynamic sensor circuit 505 during NSR, such as the intracardiac impedance signal 205 or dZ/dt signal 215 in FIG. 2. In some examples, the hemodynamic signal template is determined using a snapshot of the signal sampled at a time when the subject is in NSR. An approach for generating electrical cardiac signal templates using a snapshot of the subject's conducted heart beats are described in Kim et al., U.S. Pat. No. 6,708,058, entitled “Normal Cardiac Rhythm Template Generation System and Method,” filed Apr. 30, 2001, which is incorporated herein by reference.

In some examples the hemodynamic signal template may be a determined from central tendency (e.g., an average) of several signal snap shots to reduce signal noise. Subsequently sensed hemodynamic signals are then used to determine whether the hemodynamic signal template represents NSR.

In some examples, the signal analyzer module 520 determines a correlation value to indicate whether the hemodynamic signal correlates to the hemodynamic template signal. The signal analyzer module 520 then determines a measure of variability of the correlation value. The correlation value (e.g., a feature correlation coefficient) can provide an indication of a degree of similarity between the shape of a depolarization being examined and the shape of the hemodynamic signal template. The correlation value can be compared to a correlation threshold value (e.g., a threshold feature correlation coefficient value) to determine whether the sensed hemodynamic signal is representative of NSR. Descriptions of determining morphological correlation values is found in Kim et al., U.S. patent application Ser. No. 11/749,283, “Self-Adjusting Morphological Feature Correlation Threshold,” which is incorporated herein in its entirety. When the variability of the correlation value is determined, the rhythm discrimination module 525 deems whether the onset of tachyarrhythmia is indicative of ventricular tachycardia according to the determined variability.

In some examples, the IMD 500 creates the template used in the comparison, and in some examples, the IMD 500 receives the template from an external device and then stores the received template.

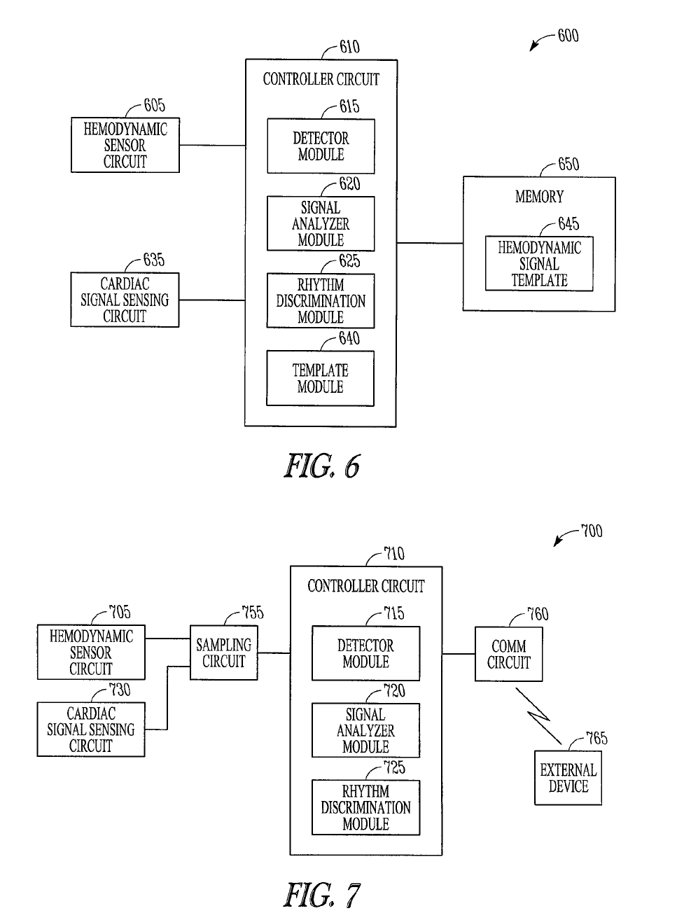

FIG. 6 shows a block diagram of another example of an IMD 600 that identifies tachyarrhythmia. The IMD 600 is configured to create a hemodynamic signal template. The IMD 600 includes an implantable hemodynamic sensor circuit 605, and a controller circuit 610. The controller circuit 610 includes a detection module 615, a signal analyzer module 620, and a rhythm discrimination module 625.

The IMD 600 also includes an implantable cardiac signal sensing circuit 635 communicatively coupled to the controller circuit 610. The implantable cardiac signal sensing circuit 635 obtains a sensed electrical cardiac signal associated with an action potential of the patient's heart. Examples of cardiac signal sensing circuits include, among other things, a subcutaneous ECG sensing circuit, an intracardiac electro-gram (EGM) sensing circuit, and a wireless ECG sensing circuit. In a subcutaneous ECG sensing circuit, electrodes are implanted beneath the skin and the ECG signal obtained is referred to as subcutaneous ECG or far-field electro-gram. In an intracardiac EGM circuit, at least one electrode is placed in or around the heart. A wireless ECG includes a plurality of electrodes to provide differential sensing of cardiac signals to approximate a surface ECG. Descriptions of wireless ECG systems are found in commonly assigned, co-pending U.S. patent application Ser. No. 10/795,126 by McCabe et al., entitled “Wireless ECG in Implantable Devices,” filed on Mar. 5, 2004, which is incorporated herein by reference in its entirety.

The controller circuit 610 includes a template module 640 to determine a fiducial identifier for alignment of a plurality of sensed hemodynamic signals using the sensed electrical cardiac signal, and to generate the hemodynamic signal template using a central tendency of aligned sensed hemodynamic signals. The generated hemodynamic signal template 645 would then be stored in a memory 650 integral to, or communicatively coupled to, the controller circuit 610. As an illustrative example, the template module 640 may use an R-wave 220A in the sensed ECG signal 210 of FIG. 2 as a fiducial identifier to align a plurality of sensed intracardiac impedance signals and generate a template of the intracardiac impedance signal using an average of the aligned signals. Variability of a subsequently sensed hemodynamic signal would then be determined by a comparison to the stored hemodynamic signal template 645.

In some embodiments, the template module 640 discriminates among candidate sensed hemodynamic signal segments for inclusion in the signal template. The template module 640 identifies a candidate hemodynamic signal for inclusion in the template generating process using at least one rule. For example, the rule could identify a candidate signal segment based on an existence or prevalence of the chosen fiducial identifier. The template module 640 then discriminates, according to the rule, whether the candidate signal segment is representative of cardiac mechanical function during an intrinsic depolarization.

For example, the rule may discriminate to only allow candidate signal segments that are representative of the selected cardiac mechanical function during NSR to be selected for inclusion in the template. In another example, the rule may only allow selection of hemodynamic signal segments that occur during SVT for inclusion in the template. This is useful in generating a template for identification of SVT to discriminate between VT and SVT.

As described previously, in some examples, the hemodynamic signal template is established by an external device, and the external device downloads the signal template to the IMD for storage. Variability of a subsequently sensed hemodynamic signal is determined by a comparison to the hemodynamic signal template received into the IMD.

FIG. 7 shows a block diagram of still another example of an IMD 700 that identifies tachyarrhythmia. The IMD 700 includes an implantable hemodynamic sensor circuit 705, and a controller circuit 710. The controller circuit 710 includes a detection module 715, a signal analyzer module 720, and a rhythm discrimination module 725. The IMD 700 also includes an implantable cardiac signal sensing circuit 730 communicatively coupled to the controller circuit 710. The implantable cardiac signal sensing circuit 730 obtains a sensed electrical cardiac signal associated with an action potential of the patient's heart.

The IMD 700 also includes a sampling circuit 755 to obtain sampled data from a sensed hemodynamic signal and from the sensed electrical cardiac signal. The IMD 700 includes a communication circuit 760 for communicating information with an external device 765. The communication circuit 760 may include a near field antenna for near-field wireless telemetry communication, and/or may include a far field antenna for far-field wireless telemetry communication.

The controller circuit 710 is communicatively coupled to the sampling circuit 755 and the communication circuit 760. The controller circuit 710 communicates the sampled data to the external device 765. The sampled data may include samples of the sensed hemodynamic signal and/or samples of the sensed electrical cardiac signal. The controller circuit 710 receives the established hemodynamic signal template from the external device 765 via the communication circuit 760. In some examples, the hemodynamic signal template is stored in a memory. The external device may establish the hemodynamic signal template using a snapshot of a sensed hemodynamic signal, or may establish the hemodynamic signal template from multiple segments of the hemodynamic signal.

Returning to FIG. 5, in some examples, the IMD 500 includes a therapy circuit 570 communicatively coupled to the controller circuit 510. The therapy circuit 570 provides at least one of pacing therapy, cardioversion therapy, or defibrillation therapy. If the rhythm discrimination module declares that the detected the tachyarrhythmia episode is indicative of VT, the controller circuit 510 may initiate cardioversion or defibrillation therapy.

According to some embodiments, the rhythm discrimination module 525 deems whether the onset of tachyarrhythmia (e.g., a rate increase) is indicative of VT or indicative of SVT according to the measure of variability. SVT often includes a relatively organized atrial-ventricular (AV) synchrony. For this reason, an SVT rhythm may show more regularity and less variability than a VT rhythm where a greater amount of AV dyssynchrony is likely to be present. If the onset is deemed to be VT, the controller circuit 510 initiates at least one of ATP therapy, cardioversion therapy, or defibrillation therapy, according to the measure of morphological variability. If the onset is deemed to be indicative of SVT, the controller circuit 510 may not initiate therapy.

The devices and methods described herein may be used alone to identify and categorize tachyarrhythmia, or may be used in conjunction with electro-gram or ECG based methods to provide enhancement to tachyarrhythmia rhythm discrimination.

The above detailed description includes references to the accompanying drawings, which form a part of the detailed description. The drawings show, by way of illustration, specific embodiments in which the invention can be practiced. These embodiments are also referred to herein as “examples.” All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAM's), read only memories (ROM's), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
an implantable hemodynamic sensor circuit, wherein the hemodynamic sensor circuit provides a hemodynamic signal, other than an intrinsic electrical cardiac signal, and wherein the hemodynamic signal is representative of mechanical function of a cardiovascular system of a subject; and
a controller circuit, communicatively coupled to the hemodynamic sensor circuit, wherein the controller circuit includes:
a detection module configured to detect an onset of an episode of tachyarrhythmia;
a signal analyzer module configured to determine a measure of morphological variability of the hemodynamic signal during the episode of tachyarrhythmia; and
a rhythm discrimination module configured to use the measure of morphological variability to derive an indication of whether the tachyarrhythmia episode is indicative of ventricular tachycardia (VT).

2. The apparatus of claim 1, wherein the signal analyzer module comprises a comparison module and a hemodynamic signal template and is configured to:
compare a morphological feature of the sensed hemodynamic signal to the hemodynamic signal template; and
determine the measure of morphological variability of the hemodynamic signal using the comparison.

3. The apparatus of claim 2, wherein the signal analyzer module is configured to:
use the comparison module to determine a correlation value that indicates whether the hemodynamic signal correlates to the hemodynamic signal template;
determine the measure of morphological variability using the correlation value, and
wherein the rhythm discrimination module is configured to derive the indication of whether the tachyarrhythmia episode is indicative of ventricular tachycardia using the measure of morphological variability obtained using the correlation value.

4. The apparatus of claim 2, including:
an implantable intrinsic electrical cardiac signal sensing circuit, communicatively coupled to the controller circuit, configured to obtain a sensed intrinsic electrical cardiac signal associated with an action potential of the subject's heart, and
wherein the controller circuit includes a template module configured to:
determine a fiducial identifier for alignment of a plurality of sensed hemodynamic signals using the sensed intrinsic electrical cardiac signal; and
generate the hemodynamic signal template using a central tendency of aligned sensed hemodynamic signals.

5. The apparatus of claim 4, wherein the template module is configured to:
identify a candidate hemodynamic signal, for inclusion in the template generating, using at least one rule; and
determine, according to the rule, whether the candidate hemodynamic signal is representative of cardiac mechanical function during an intrinsic depolarization activated heart contraction.

6. The apparatus of claim 2, including:
an implantable intrinsic electrical cardiac signal sensing circuit, communicatively coupled to the controller circuit, configured to obtain a sensed intrinsic electrical cardiac signal associated with an action potential of the subject's heart;
a sampling circuit, configured to obtain sampled data from the sensed hemodynamic signal and the sensed intrinsic electrical cardiac signal; and
a communication circuit, configured for communicating information with an external device,
wherein the controller circuit is communicatively coupled to the sampling circuit and the communication circuit and is configured to:
communicate the sampled data to the external device via the communication circuit; and
receive the hemodynamic signal template from the external device via the communication circuit.

7. The apparatus of claim 1, wherein the signal analyzer module is configured to:
determine a measure of variability of a time duration between two different fiducial identifiers in the hemodynamic signal;
determine a measure of morphological complexity of the hemodynamic signal during the episode of tachyarrhythmia; and
wherein the rhythm discrimination module is configured to deem whether the tachyarrhythmia is indicative of ventricular tachycardia using as the measure of morphological variability both the measure of variability of the time duration and the measure of morphological complexity.

8. The apparatus of claim 1, wherein the signal analyzer module is configured to measure morphological variability of the hemodynamic signal during the episode of tachyarrhythmia using of at least one of:
a maximum of the hemodynamic signal,
a minimum of the hemodynamic signal,
a maximum rate of change of the hemodynamic signal,
a minimum rate of change of the hemodynamic signal,
a time when the hemodynamic signal reaches a specified maximum,
a time when the hemodynamic signal reaches a specified minimum,
a time of occurrence of an inflection point in the hemodynamic signal,
an area under a curve of a segment of the hemodynamic signal, or
an Nth moment of the hemodynamic signal, wherein N is a specified value.

9. The apparatus of claim 1, wherein the signal analyzer module is configured to:
determine a first fiducial identifier and a second fiducial identifier in the hemodynamic signal; and
obtain the measure of morphological variability using a measure of variability of a time duration between the first and second fiducial identifiers.

10. The apparatus of claim 1, wherein the signal analyzer module comprises a time-to-frequency domain transformation module and is configured to:
obtain a transform of the hemodynamic signal from a time domain to a frequency domain; and
measure the morphological variability by measuring a variability of at least one of a dominant frequency of the transformed hemodynamic signal, or a magnitude of a power spectral density of the transformed hemodynamic signal.

11. The apparatus of claim 1 including:
an implantable intrinsic electrical cardiac depolarization sensing circuit, communicatively coupled to the controller circuit, configured to obtain a sensed intrinsic electrical cardiac depolarization signal from a ventricle, and
wherein the detection module is configured to:
monitor a rate of ventricular depolarizations of the subject using the sensed intrinsic electrical cardiac depolarization signal; and
detect the onset of the tachyarrhythmia episode upon detecting a rate of ventricular depolarizations that exceeds a ventricular tachycardia detection rate threshold value for a specified time duration.

12. The apparatus of claim 1, wherein the hemodynamic sensor circuit includes an intracardiac impedance sensing circuit, and wherein the hemodynamic signal is representative of intracardiac impedance of the subject.

13. The apparatus of claim 1, wherein the hemodynamic sensor circuit includes a transthoracic impedance sensing circuit, and wherein the hemodynamic signal is representative of a cardiac component of transthoracic impedance of the subject.

14. The apparatus of claim 1, wherein the hemodynamic sensor circuit includes a blood pressure sensing circuit, and wherein the hemodynamic signal is representative of blood pressure.

15. The apparatus of claim 14, wherein the blood pressure sensing circuit includes at least one of:
a pressure sensor configured to be implanted in a coronary vessel to determine left ventricle pressure by direct measurement of a coronary vessel pressure,
a right ventricle (RV) chamber pressure sensor, configured to be located in a right ventricle of the subject,
a pulmonary artery pressure sensor, configured to be located in a pulmonary artery of the subject,
a left atrial chamber pressure sensor, configured to be located in a left atrium of the subject, or
a pressure sensor that is configured to be implanted in a vein near an artery to determine arterial pressure from contact with the artery.

16. The apparatus of claim 1, wherein the hemodynamic sensor circuit includes at least one of: a heart sound sensor, a cardiac wall motion sensor, or a supracardiac impedance sensing circuit.

17. The apparatus of claim 1, including a therapy circuit, communicatively coupled to the controller circuit, configured to provide at least one of pacing-level electrostimulation therapy, cardioversion therapy, or defibrillation therapy,
wherein the rhythm discrimination module is configured to use the measure of morphological variability to derive an indication of whether the onset of the episode of tachyarrhythmia is indicative of VT or supraventricular tachycardia (SVT), and
wherein, when the onset of the episode of tachyarrhythmia is deemed to be VT, the controller circuit is configured to initiate at least one of anti-tachycardia pacing (ATP) therapy, cardioversion therapy, or defibrillation therapy, using the measure of morphological variability to select the therapy to be initiated.

18. A method comprising:
sensing a hemodynamic signal, other than an intrinsic electrical signal, using an implantable sensor, wherein the hemodynamic signal comprises a signal representative of mechanical function of a cardiovascular system of a subject;
detecting an onset of an episode of tachyarrhythmia;
determining a measure of morphological variability of the hemodynamic signal during the episode of tachyarrhythmia; and
deriving an indication of whether the onset of the episode of tachyarrhythmia is indicative of VT by using the measure of morphological variability.

19. The method of claim 18, wherein determining a measure of morphological variability includes:
comparing a morphological feature of the sensed hemodynamic signal to a hemodynamic signal template; and
determining the measure of morphological variability of the hemodynamic signal during the onset of the episode of tachyarrhythmia using the comparison.

20. The method of claim 19, wherein determining a measure of morphological variability includes:
determining a correlation value to indicate whether the hemodynamic signal correlates to the hemodynamic signal template;
determining a measure of variability of the correlation value; and
deriving the indication of whether the onset of the episode of tachyarrhythmia is indicative of ventricular tachycardia using the measure of variability of the correlation value.

21. The method of claim 19, including:
sensing an intrinsic electrical cardiac signal associated with an action potential of the subject's heart;
determining a fiducial identifier, for alignment of a plurality of sensed hemodynamic signals, using the sensed electrical cardiac signal; and
generating the hemodynamic signal template using a central tendency of aligned sensed hemodynamic signals.

22. The method of claim 21, including:
identifying a candidate hemodynamic signal, using at least one rule, for inclusion in generating the hemodynamic signal template; and
using the rule to determine whether the candidate hemodynamic signal is representative of cardiac mechanical function during an intrinsic depolarization activated heart contraction.

23. The method of claim 18, wherein determining a measure of morphological variability includes:
determining a measure of variability of a cycle length of the hemodynamic signal;
determining a measure of morphological complexity of the hemodynamic signal during the episode of tachyarrhythmia; and
deriving the indication of whether the onset of the episode of tachyarrhythmia is indicative of ventricular tachycardia using both the measure of variability of the cycle length and the measure of morphological complexity.

24. The method of claim 18, wherein detecting a tachyarrhythmia episode includes:
monitoring a rate of ventricular contractions of the subject; and
detecting that an increase in the rate of ventricular contractions exceeds a ventricular tachycardia detection rate threshold value for a specified time duration.

25. The method of claim 18, wherein sensing a hemodynamic signal includes sensing a signal representative of at least one of intracardiac impedance or a cardiac component of transthoracic impedance.

26. The method of claim 18, wherein sensing a hemodynamic signal includes sensing a signal representative of blood pressure.

27. The method of claim 26, wherein sensing a signal representative of blood pressure includes sensing a signal representative of at least one of:
right ventricular pressure,
pulmonary arterial pressure,
right atrial pressure,
left atrial pressure,
left ventricular pressure,
left ventricular pressure as sensed in a coronary vessel, or
an arterial pressure.

28. The method of claim 18, wherein sensing a hemodynamic signal includes sensing at least one of:
a heart sound signal associated with activity of the subject's heart,
a ventricular wall motion signal representative of ventricular wall motion, or
a supracardiac impedance signal representative of supracardiac impedance.

29. The method of claim 18, wherein determining a measure of morphological variability includes measuring variability of a hemodynamic signal feature of the hemodynamic signal, wherein the hemodynamic signal feature includes at least one of:
a maximum of the hemodynamic signal,
a minimum of the hemodynamic signal,
a maximum rate of change of the hemodynamic signal,
a minimum rate of change of the hemodynamic signal,
a time when the hemodynamic signal reaches a specified maximum value,
a time when the hemodynamic signal reaches a specified minimum value,
a time of occurrence of an inflection point in the hemodynamic signal,
an area under a curve identified in a segment of the hemodynamic signal, or
an Nth moment of the hemodynamic signal, wherein N is a specified value.

30. The method of claim 18, wherein determining a measure of morphological variability includes:
determining a first fiducial identifier and a second fiducial identifier in the sensed hemodynamic signals; and
determining the morphological variability by measuring a variability of a time duration between the first and second fiducial identifiers.

31. The method of claim 18, wherein determining a measure of morphological variability includes:
obtaining a frequency domain transformed hemodynamic signal; and
determining the morphological variability by using a measure of variability of at least one of a dominant frequency of the transformed hemodynamic signal, or a magnitude of a power spectral density of the transformed hemodynamic signal.

32. The method of claim 18,
wherein deriving the indication of whether the onset of the episode of tachyarrhythmia is indicative of VT includes discriminating whether the onset of the episode of tachyarrhythmia is indicative of VT or SVT, and
wherein the method includes initiating ATP therapy when the tachyarrhythmia episode is deemed to be SVT and initiating at least one of cardioversion therapy or defibrillation therapy when the tachyarrhythmia episode is deemed to be VT.
